(19) United States
(12) Patent Application Publication
(10) Pub. No.: US 2026/0158492 A1
(43) Pub. Date: Jun. 11, 2026

(54) At-Home Covid Nucleic Acid Detection Kit For Rapid Molecular Diagnostics

(71) Applicant: Rula Abbud-Antaki, Ithaca, NY (US)

(72) Inventor: Rula Abbud-Antaki, Ithaca, NY (US)

(21) Appl. No.: 19/341,197

(22) Filed: Sep. 26, 2025

Related U.S. Application Data
(62) Division of application No. 18/190,927, filed on Mar. 27, 2023.
(60) Provisional application No. 63/323,838, filed on Mar. 25, 2022.

(51) Int. Cl. B01L 3/00; C12N 15/10; G01N 21/84; G01N 33/53; G01N 33/543; G01N 33/58

(52) U.S. Cl. CPC B01L 3/502761; C12N 15/1003; G01N 21/8483; G01N 33/5308; G01N 33/54388; G01N 33/582; B01L 2200/027; B01L 2200/0647; B01L 2200/12; B01L 2200/16; B01L 2300/0822; G01N 2470/04

(57) ABSTRACT

The present invention describes kits and methods for rapid detection of nucleic acid sequences in real-time and in any setting. Unique features of this kit include isothermal nucleic acid extraction in Unit 1, and amplification, labeling, and signal detection in Unit 2 (FIG. 1). These units can be operated separately with manual transfer of product from one to the other or can be put together in a fashion where user places sample into inlet of Unit 1 and observes results in detection chamber of Unit 2. Several versions of this kit can be developed using different sequence-specific primers and probes to detect any nucleic acid sequence (DNA or RNA) in a multiplex fashion. In a preferred embodiment, visual detection of amplified signal is done using Lateral Flow Strips, eliminating the need for any additional complex equipment. Such a kit can be easily developed and manufactured to assist in the mitigation response to any pandemic, especially for COVID-19. The COVID-19 kit is called an At-Home COVID test (AH-COV).

### THE CROSS REFERENCE TO RELATED APPLICATIONS

The present application is a divisional of U.S. application Ser. No. 18/190,927, filed Mar. 27, 2023, which claims priority to U.S. Provisional Application No. 63/323,838, filed Mar. 25, 2022, both of which are incorporated herein by reference in their entirety.

### FIELD OF THE INVENTION

The present invention relates to molecular diagnostics and genomic research tools that meet the WHO REASSURED criteria for the developed world. Described herein are kits, methods, and reagents/compositions for all-inclusive, low complexity, portable, disposable, easy-to-use, and rapid detection of nucleic acids (NA) at point-of-care, home, or in the field.

### BACKGROUND OF THE INVENTION

During the recent COVID-19 pandemic, molecular diagnostics, especially quantitative RT-PCR, were at the forefront of the fight against the COVID-19 virus (SARS-CoV-2). Across the globe people lined up in their cars to get their nasal (N) or nasopharyngeal (NP) swabs taken to determine whether they were infected. However, results took several days, because samples had to be sent to laboratories that are certified under the Clinical Laboratory Improvement Amendment (CLIA) for high complexity testing.

Since it was not possible to know who is infected in real time, major lockdowns became necessary to stop the spread of the virus, especially when healthcare facilities became overwhelmed with sick patients. To overcome this bottleneck, Antigen tests, which are cheaper to make, and give immediate results became popular, but suffered from lower sensitivity leading to false negative results. These tests did not meet the REASSURED criteria for diagnostic tests (Land et al., n.d.), which state that important features of diagnostics for the developed world have to be: Real-time connectivity, Ease of sample collection, Affordable, Sensitive, Specific, User-friendly, Rapid, Equipment-free, and Deliverable to end-user.

Several companies have tried to make rapid molecular COVID tests that can be used at home. These tests detect viral RNA using isothermal nucleic acid amplification chemistries that do not rely on PCR. While these technologies addressed a need for alternatives to qRT-PCR due to reagents and instruments shortages, they have yet to reach the same limit of detection as qRT-PCR. (Barreda-García et al., 2018; Biyani et al., 2021; Ganguli et al., n.d.; Tanner et al., 2015; Zhang & Tanner, 2017)

There are only a few FDA-approved for EUA rapid molecular COVID tests, which can give results within 10 minutes to an hour. These include: the Sherlock CRISPR SARS-CoV-2 Kit, Cue COVID-19 Test, ID NOW COVID-19, iAMP COVID-19 Detection Kit, LumiraDx SARS-CoV-2 RNA STAR Complete, Detect™ Covid-19 Test, and Lucira COVID-19 All-In-One Test Kit. These tests are only authorized for use by CLIA approved laboratories, except for the Lucira test.

The reason these tests are rapid is because they rely on isothermal NA amplification, as opposed to the cycles of rapid temperature change (30 on average) needed for PCR. There are several ways to achieve target labeling and amplification without a PCR machine. The Abbott ID NOW COVID-19 test, which gives results within 5-10 minutes, relies on strand-displacing DNA Polymerase (SDDP) and DNA nicking enzyme technique: Nicking and Extension Amplification Reaction (NEAR). However, this assay has suffered from low sensitivity, leading to false negatives. In addition, it requires a docking station to run the assay and read the results, which limits its use to Point-of-Care (POC).

On the other hand, the Lucira COVID-19 All-In-One Test Kit is the only one that is approved for prescription in-home use, or at POC, using a nasal swab collected by the patient. This platform depends on Reverse Transcriptase and Loop-Mediated Isothermal Amplification (RT-LAMP), a method that also uses SDDP to give results within 30 minutes. While RT-LAMP is advantageous because it does not require NA extraction, the need for 6 different 40-45 base primers per target, incubation at 65° C., and the need for sensors to detect the halochromatic agent signal increase the cost of manufacturing. A problem that could be detrimental for wide assay adoption during a pandemic, especially when there are issues with supply chains. Another limitation of this test is its high Limit of Detection (LOD: 2700 copies per swab), making it less sensitive than qRT-PCR.

Therefore, there is an urgent need to develop highly sensitive molecular methods capable of giving rapid results, in real-time, without the need of complicated equipment, or specialized training, for mitigation of viral spread during a pandemic. Rapid molecular diagnostics tests for a variety of diseases that are not laboratory-based and can be purchased over the counter (OTC) will change patient care. They will make early detection of a variety of molecular targets possible at POC, home, or in any setting.

### SUMMARY OF THE INVENTION

The present invention consists of a kit for rapid multi-gene detection that is disposable, portable, and easy-to-use by a lay person in any setting. Disclosed herein are kits for DNA or RNA detection that consist of two complementary units: one for nucleic acid (NA) extraction and purification (Unit 1), and the other for isothermal amplification, labeling, and detection of target genes (Unit 2). A sample, consisting of patient swab, tissue, or body fluids is introduced into Unit 1, and the results appear in the detection chamber of Unit 2.

When this kit is used for detection of SARS-CoV-2 viral RNA, it is called At-Home COVID test kit (AH-COV), with AH-COV-Unit-1 being Unit 1 where patient swab is introduced, and AH-COV-Unit-2 being Unit 2 where viral RNA signal is amplified and detected.

Several kit types can be made depending on the chemistry used for NA extraction, amplification, labeling, and detection. Disclosed herein are reagents and methods for NA extraction in Unit 1, as well as methods for delivering extracted NA into Unit 2. Extraction can be performed in a tube containing lysis and extraction reagents separate from Unit 2 (Unit 1-S; AH-COV-Unit-1-S) and a dipstick or dropper can be used to transfer NA into Unit 2.

In another embodiment, Unit 1 can be made out of a cartridge with an inlet and outlet that is docked over the inlet of Unit 2. This is called Unit-1-D (docked), also called AH-COV-Unit-1-D for COVID testing. In this setting, the sample swab is introduced into Unit-1-D and extracted RNA will be pushed into the system after a brief incubation period. In some instances, purified NAs can be directly injected into Unit-2 inlet.

In another embodiment, a kit with Unit-2 is designed with several microfluidic channels that contain different gene-specific primers and controls, for portable multiplex detection of genetic markers. In addition, different enzyme chemistries for isothermal amplification and synthesis of labeled NA are disclosed. These include commonly used chemistries for isothermal NA amplification or a novel approach that uses RdRP Replicase for RNA labeling and amplification at 30° C. Depending on temperature requirement a heating element can be included in Unit 2.

Amplified NAs that come out of the microfluidic channels on Unit 2, can be pipetted out and analyzed by a variety of methods including the fast DNAq-Card from accel diagnostics, which takes less than 2 minutes. This type of device is called Unit-2-B, for basic as it does not require a label for the nascent DNA.

Also disclosed are kits that differ based on methods used to label amplified NAs. These include FITC/FAM, Biotin, Digoxigenin, Texas Red, ROX, and other commonly available colorimetric labeling tags, which can be included in the isothermal enzymatic amplification mix in Unit 2. Unit-2 can be retrofitted with Whatman paper that have lines filled with signal visualization antibodies and chemistries. These papers soak up nascent labeled NAs and allow for colorimetric visualization of signal. These are Unit-2-LF for lateral flow. In the case of AH-COV, this kit is called AH-COV-LF.

Another version of the device uses fluorometric real-time monitoring of signal using fluorescence-based labeling chemistry and is called Unit-2-Fluor; AH-COV-Fluor for COVID. Visualization and measurement of signal requires use of commonly available fluorescence visualization devices that can be lab-based or portable.

Different analytical and clinical validation approaches can be done using any combination of the abovementioned devices, chemistries, and visualization methods to make this rapid NA detection device useful for early detection and diagnosis of a variety of human, animal, or plant diseases, or in any setting where rapid on-the-spot detection of NAs is required.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A fuller understanding of the invention can be gained from the following description when read in conjunction with the accompanying drawings, in which:

FIG. 1 is a flow chart describing the essential components of the device according to the present invention;

FIGS. 2A and 2B depict the procedure for introducing the Sample into AH-COV-Unit-1. Shown are two envisioned means, and associated embodiments, for introducing the Sample. The preferred embodiment is provided in FIG. 2A, wherein the sample is introduced via a swab into a cartridge that docks on to the inlet of AH-COV-Unit-2. FIG. 2B provides an alternate embodiment AH-COV-Unit-1-S, wherein a separate test tube is used to extract the NA from the sample, and a dipstick transfers the extracted NA to AH-COV-Unit-2;

FIGS. 3A, 3B and 3C depict three different embodiments of AH-COV-Unit-2 comprising of a microfluidic slide with three different target amplification and detection means: AH-COV-Unit-2-B (Basic; FIGS. 3A, 300), AH-COV-Unit-2-LF (Lateral Flow; FIGS. 3B, 310), and AH-COV-Unit-2-Fluor (Fluorescence; FIGS. 3C, 320);

FIG. 4A shows an AH-COV-Unit-2.1 30 μm (left) and a 100 μm (right) loaded with red food coloring; and FIG. 4B is a photograph of agarose gels showing amplified Positive DNA Control, on-chip and off-chip, at 1:10, 1:100, but not at 1:1000;

FIG. 5 shows a quantitative measurement of an RPA product obtained on an AH-COV-Unit-2.1, using a DNAq-Card™ (Accel Diagnostics);

FIGS. 6A and 6B show mixing of three liquids on AH-COV Unit-2.2. FIG. 6A is an image of the device at 15 minutes after consecutive addition of yellow (10 μl), red (2.5 μl), and blue (10 μl) dyes in each inlet port; and FIG. 6B is an image at 30 minutes; and

FIG. 7 shows AH-COV-Unit-2 configured for detection of four targets from the same sample simultaneously. Four serpentine channels emerge from one sample inlet. Primer and amplification mixes for different targets can be preloaded and dried in each channel, and the signal can be detected on four individual lateral flow strips provided at the exit of each of the serpentine channels at each corner of the device.

### DETAILED DESCRIPTION OF THE INVENTION

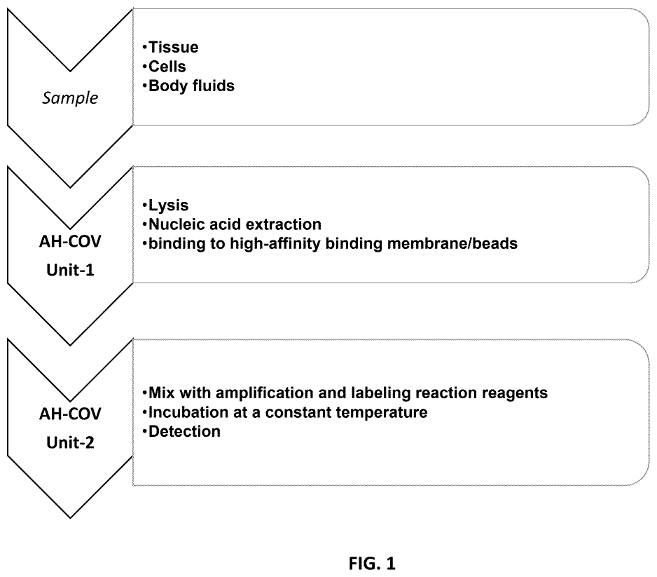

The present invention provides a handheld, disposable, easy-to-use, rapid nucleic acid (NA) detection kit for use in any setting. It contains an all-in-one isothermal NA extraction, purification, amplification, labeling, and detection device, packaged together with sample collection tools (such as swabs, droppers, or other sample collection means), reagents, and instruction manual for use by a person 13 years or older (see FIG. 1).

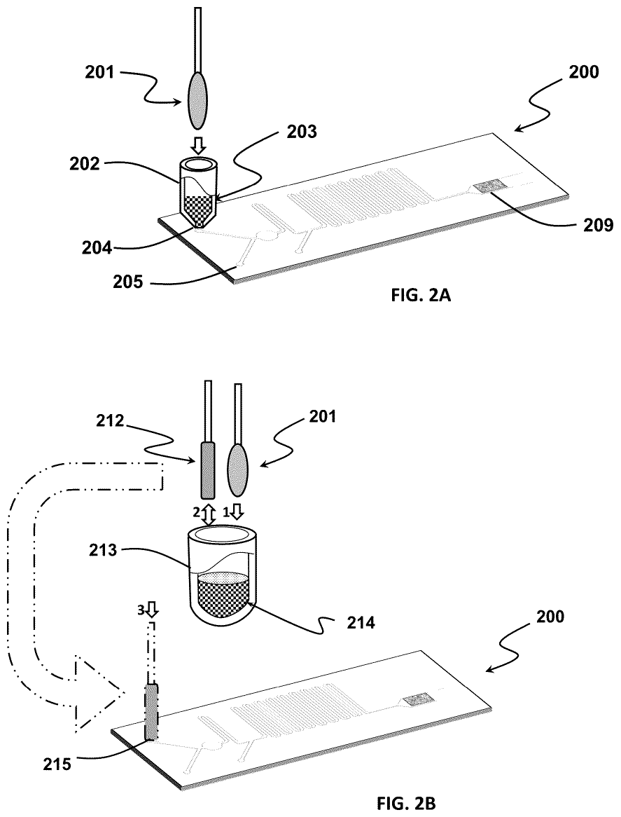

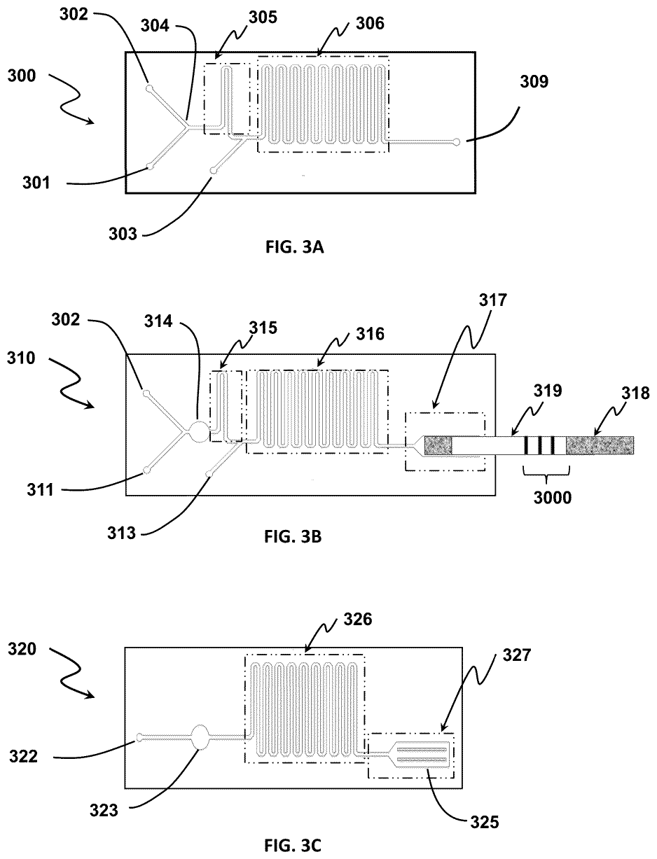

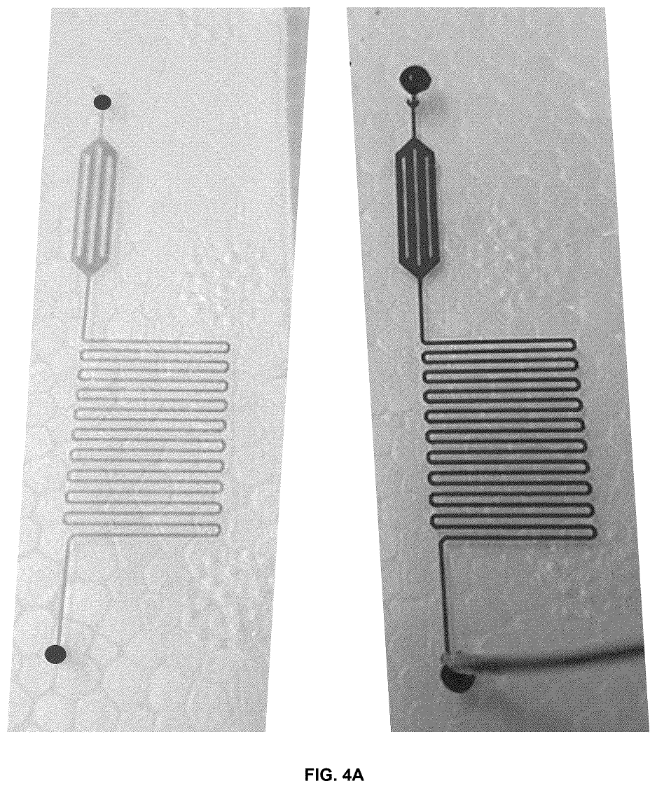

Unique features of the device include: (1) a sample docking unit (Unit-1) for NA extraction from raw materials (FIG. 2); (2) a microfluidic chip (Unit-2) with an inlet for docking Unit-1; and release of extracted NA (FIG. 3). In addition, Unit-2 has microchannels for mixing sample RNA with amplification and labeling reaction reagents, serpentine channels for incubation, and a chamber for signal detection.

When in use, a sample, comprised of Tissue, Cells, or Bodily Fluids, is introduced into a first unit (Unit-1) of the device, wherein lysis of the sample occurs and nucleic acids are extracted and bound to a high-affinity nucleic acid binding solid support. Thereafter, the sample is transferred into a second unit (Unit-2), where it is mixed with amplification and labeling reagents, and incubated at a constant temperature for rapid visualization and detection of target sequence using a variety of colorimetric and fluorescence methods, depending on the type of label used in the reaction.

In some embodiments, the device is called AH-COV and contains oligonucleotide primers specific for isothermal amplification of SARS-CoV-2 RNA, the virus responsible for COVID-19. Patient swabs or saliva are introduced in AH-COV-Unit-1 and results are read on AH-COV-Unit-2, either with the naked eye or using a smartphone-based application.

Assay sensitivity and specificity depend on 1) sufficient RNA yield, 2) isothermal labeling and amplification of target RNA, and 3) detection of signal generated by labeled target. For most COVID-19 diagnostics protocols, patient swabs are placed in Viral Transport Medium (VTM) and sent out to the lab, where viral RNA is detected by qRT-PCR. For AH-COV, the patient swab is immediately placed in the device's AH-COV-Unit-1. Depending on the amplification enzymes used in AH-COV-Unit-2, ultra-pure RNA can not be needed. Therefore, we have designed AH-COV so that AH-COV-Unit-1 can be separate from AH-COV-Unit-2, allowing for independent evaluation of the analytical performance of each unit.

Referring to FIG. 2, disclosed are two embodiments of AH-COV-Unit-1. The first embodiment provided in FIG. 2A consists of a cartridge 203 configured to receive a sample swab 201 and docks directly on the sample inlet 204 of Unit-2 200. Contained in cartridge 203 is a quantity of lysis and extraction buffer 203 for inactivation of the virus and extraction of NA at room temperature. Sample RNA is bound to a high affinity NA binding membrane or beads in 204 get mechanically released and flushed with a wash buffer into AH-COV-Unit-2 200.

In a second embodiment, the user is provided a separate test tube 213 containing lysis and extraction buffer 214 configured to receive sample swab 201 and a NA binding dipstick 212. The swab is inserted into the lysis and extraction buffer and remains in place. The dipstick 212 is dipped into the solution and then removed, whereupon it transfers the bound NA to sample inlet 215 of AH-COV-Unit-2 200.

Referring to FIG. 3, three different embodiments of an AH-COV-Unit-2 300, 310, 320 are depicted. It comprises a microfluidic device that is approximately 1″×3″ with inlets for the sample 302 and amplification reaction reagents 301, 311; serpentine channels for mixing 305, 315 and incubation 306, 316, 326; and one outlet for detection of amplified product 309, 317, 327. In some instances, the sample and amplification reaction mix are added simultaneously, allowed to mix in the mixing channels, and followed by Magnesium Acetate (MgOAc, 303, 313) to activate the enzymatic reaction (FIG. 3A, B). In other instances, the amplification reaction mix is dried in a chamber 323 on the chip during fabrication (FIG. 3C) and the activating reagents are added after sample introduction in the inlet 322. This reaction incubates inside the serpentine channels, which have a length proportional to the time needed for appearance of the amplified product.

Depending on the enzymatic reactions used for isothermal target amplification and labeling, a heating element can be incorporated into Unit 2 of the device. Preferred embodiments are using RPA (Recombinase Polymerase Amplification) run at 37° C., while Phi29 Polymerase and Replicase (RdRP) work well at 30° C.

Signal detection on AH-COV-Unit-2 differs based on the number of targets tested, type of probes, and amplification reaction used. AH-COV-Unit-2-B (FIG. 3A) is used when amplified product needs to be collected at the end of the reaction for further downstream testing by gel electrophoresis or on DNAq-Cards™ (Accell Dx). On the other hand, AH-COV-Unit-2-LF (FIG. 3B) is used whenever two targets are labeled with either biotin and digoxigenin, or biotin and FITC/FAM. For this device, the detection chamber is retrofitted with lateral flow strips that have a control line, and two test lines, one for the target and the other for the internal control. In other instances, multiple targets can be labeled with different fluorophores and signal is detected in real-time using a fluorometer (FIG. 3C). This device is called an AH-COV-Unit-2-Fluor and can be used for real-time monitoring and measurement of the amplified product.

In an embodiment of this invention, establishment of the analytical and clinical validity of the assay on AH-COV will require optimization of: (1) NA extraction and purification from patient swabs using AH-COV-Unit-1; (2) isothermal labeling and signal detection of multiple target genes on AH-COV-Unit-2; and (3) and putting both units together for testing of samples obtained from infected and uninfected individuals.

### NA Extraction and Purification from Patient Swabs Using AH-COV-Unit-1.

For current COVID-19 molecular assays, extraction of intact, undegraded viral RNA from nasal swabs requires additional time and equipment, further delaying diagnosis. While good quality viral RNA is needed for qPCR, isothermal DNA amplification methods, such as RPA and LAMP, do not require ultrapure NAs. Cellulose or Whatman #1 paper can be used for nucleic acid purification from animal and plant cells within 30 seconds. Captured RNA can be amplified by RT-RPA and visualized by agarose gel electrophoresis.

In the present invention, faster results can be obtained because NA extraction from patient swabs is integrated within the device. It occurs in the AH-COV-Unit-1, which contains extraction and lysis buffers. In addition, NA purification is achieved through binding to a membrane or beads (AH-COV-Unit-1-D) or a dipstick (AH-COV-Unit-1-S). To evaluate the minimal requirements needed for obtaining sufficient detectable amounts of viral RNA, three different RNA purification and capture methods from nasal swabs can be used by testing three different chemistries.

For example, three different lysis buffers in combination with three different NA binding membranes or beads can be used for high RNA yield from nasal swabs obtained from three uninfected individuals. Such buffer compositions can comprise the following: (a) guanidium-based RNA isolation by placing a swab in 500 ul 1.5M Guanidium HCl, 50 mM Tris pH 8.0, 100 mM NaCl, 5 mM EDTA, 1% Tween-20, and 40 ug/ml Proteinase K; (b) mechanical disruption in 500 ul 20 mM Tris pH 8.0, 25 mM NaCl, 2.5 mM EDTA, 0.05% SDS and adding two ball bearings and shaking for eight seconds; and (c) alkaline lysis buffer: 200 mM NaOH, 60% (v/v) PEG-200, 2% (v/v) Triton X-100.

Three different types of RNA binding methods can be employed using Whatman paper #1, cellulose membrane, or silica beads. Following sample lysis and RNA binding on AH-COV-Unit-1-S, the dipstick is removed, dipped in wash buffer (10 mM Tris pH 8.0, 0.1% Tween-20), and inserted into the inlet reservoir of AH-COV-Unit-2-B, which contains RT-RPA (Twist® Amp Basic) amplification reaction reagents for RNAseP. This triggers the elution of the RNA into the solution, and amplification of the control RNAseP cDNA. Controls for these experiments include swabs that have no sample, and no RT. Furthermore, dipsticks can be placed in RT-RPA reactions in a test tube for off-chip control. Thus, for each experimental swab, four different AH-COV-Unit-2-B and 4 DNAq-Cards™ are needed. In total, this experiment requires 108 AH-COV-Unit-2-B and DNAq-Cards™ and denatured agarose gels. Off-chip experiments only are run on the gel.

Similar experiments can be performed for optimization of lysis buffer and RNA binding using AH-COV-Unit-1-D. For these experiments, the binding membrane or beads are pre-packaged in the sample port of AH-COV-Unit-2-B. First, the nasal swab is inserted into the AH-COV-Unit-1-D cartridge containing the lysis buffer. After a short incubation, the cartridge is pushed into the inlet allowing the buffer to flow through the device's microchannels. Wash buffer is pushed through the system prior to addition of (Twist® Amp Basic) amplification reaction reagents for RNAseP. Amplified product is collected and quantified using DNAq-Cards™ and agarose gels. One additional control is added to these experiments where there is no RNA binding membrane. An aliquot of the lysis buffer is pushed into AH-COV-Unit-2 at the same time as the amplification reaction reagents. Statistical comparison between the yield from the different combinations of buffers and RNA binding beads are done using ANOVA followed by student's t-test.

The enzymes used on AH-COV have been reported to be tolerant of RNA preparations that are not ultra-pure. Heat has been reported to be sufficient for cell lysis and virus inactivation prior to isothermal NA amplification. However, alkaline cell lysis, which works at room temperature, will work well, as long as it is followed with a neutralization buffer or significantly diluted, prior to addition of amplification reaction mix. This is why there is a wash step in the protocol. The outcome of these experiments determines the necessity of binding the extracted NA to membrane or beads prior to downstream amplification on AH-COV-Unit-2. The final results from these experiments deliver the optimal protocol for virus inactivation and RNA release from the swabs.

Once lysis and wash buffer composition, and NA capture membrane or beads requirement are identified, AH-COV-Unit-1 can be manufactured to either directly feed into AH-COV Unit 2 (AH-COV-Unit-1-D), or remain separate (AH-COV-Unit-1-S). Optimization of AH-COV-Unit-2 can be done independently of AH-COV-Unit-1, as it can be done using commercially available viral RNA and patient sample controls.

### Isothermal Labeling and Signal Detection of Multiple Target Genes on AH-COV-Unit-2.

Target RNA amplification usually requires making a cDNA copy of the RNA using RT followed by PCR amplification in the presence of fluorescent probes for quantitative monitoring of the amplified signal. RT and PCR require different temperature cycles and are not amenable for rapid testing. The present invention relies on isothermal DNA amplification approaches combined with rapid detection methods, allowing for rapid results with minimal instrumentation.

Several isothermal target amplification approaches can be tested on this device. These include commonly used chemistries for DNA amplification and labeling, such as RPA from Twist-Dx, LAMP from New England Biolabs (NEB), and Phi29 Polymerase (Lucigen). For isothermal RNA amplification and labeling, MMLV-RT is added to the device and the enzyme mix is RT-RPA, RT-LAMP, or RT-Phi29. These reactions require incubation at 37° C., 65° C., and 65° C. followed by 30° C., respectively. In addition, a novel approach that uses RdRP Replicase for RNA labeling and amplification at 30° C. is disclosed. While MMLV-RT and Phi29 Polymerase have been previously used for amplification of RNA, Replicase has not been used for molecular diagnostics purposes. It relies on the ability of RNA-dependent Polymerase (RdRP or Replicase) to make copies of template RNA without the need for Reverse Transcriptase (RT), making the assay go faster.

Target amplification experiments can be performed in a 0.5 ml Eppendorf tube to obtain best conditions. The Twist® Amp Basic protocol can be modified to include digoxigenin-labeled forward primers and biotin-labeled primers for RNAseP and different concentrations of control synthetic RNAseP RNA (Biosearch IDT Technologies). The amplified product can be visualized using denatured agarose gel, quantified using DNAq-card™ and tested on PCRD lateral flow strips from Abingdon Health.

For RT-Phi29, MMLV-RT (Lucigen) which has RNAse H activity can be used in combination with Phi29 Polymerase (Lucigen) to amplify and label cDNA from template RNAseP control RNA, in the presence of labeled forward and reverse primers (biotin, digoxigenin, or FITC/FAM) at room temperature. Amplified product can be examined on agarose gel, DNAq-card™, and tested on PCRD lateral flow strips.

As for the replicase amplification reaction chemistry, the RdRP from SARS-CoV-2, which has been shown to catalyze de novo RNA synthesis in a primer-dependent fashion, can be examined for its utility for isothermal target detection on AH-COV. By using biotin-labeled gene-specific primers with FAM or digoxigenin-labeled d-ATP, amplification and labeling of the signal from these genes can occur within 15-20 minutes.

In one embodiment, the Replicase reaction mixture can contain a Polymerase assay buffer (20 mM Tris, pH 8.0, 10 mMKCl, 1 mM DTT, 2 mM MgCl2), 100 nM biotin-labeled primers, 500 nM purified SARS-CoV-2 RdRP, nsp7, and nsp8 proteins, 500 μM (GTP, UTP, CTP), and 50 μM FAM or digoxigenin-labeled 3′-dATP. RdRp, nsp7, and nsp8 can be synthesized using cell-free protein synthesis systems (NEBiolabs). Replicase is able to synthesize double-labeled target RNA at 30° C. within 20-30 minutes. The labeled RNA product of this reaction can be detected with any of the detection methods described above depending on the labels used on the primers and dATP.

The assay LOD can be measured for each reaction at 10, 20, 30, and 45 minutes of incubation with the goal of obtaining results within 15-45 minutes. The outcome of these experiments give us an alternative to RPA which can be further explored in the future. Currently, we are able to test different methods to maintain the temperature of AH-COV devices that use RT-RPA. Experiments can be performed to determine whether holding the reaction tube or using a hand warmer would give similar results to using an incubator.

In another embodiment, AH-COV can be developed using RT-RPA chemistry for detection of SARS-CoV-2 genes. RPA requires several proteins, including a recombinase, a recombinase loading factor, a single strand binding protein (ssBP), and the TwistAmp DNA Polymerase, which is a SSDP. The first three proteins are needed for annealing the primers to dsDNA, and the strand displacement capability of the polymerase allows for rapid isothermal DNA synthesis. Only two primers per target are needed, as opposed to the six primers needed for LAMP, making assay development a lot easier. To achieve high levels of sensitivity and specificity for viral RNA detection, the following variables are optimized: (1) primer pairs and probes; (2) LOD; and (3) specificity. Described below is the approach used for AH-COV, and it can be applied for development of kits capable of detection of any other types of RNA, DNA, or nucleic acids.

### 1. Design Primer Pairs and Probes

The TwistAmp® Exo protocol, which allows for detection of time of amplification onset (threshold) and measurement of the total amplified signal is used for selection of primers pairs and probes for SARS-CoV-2 N, S, E, and RdRP genes. RPA requires primers that are 30-36-mers, with 40-60% GC, and very little repetitive sequences. The optimal length of the amplified product should be 100 to 200 bp. Probes are designed with a Fluorophore and quencher flanking a THF dSpacer, and a 3′ modification to block extension by polymerase. The quencher blocks fluorescence when the probe is bound to DNA, and ExoIII tries to repair the mismatch created by THF on the bound probe and cuts it, leading to separation of quencher from probe and appearance of fluorescence signal.

Several fluorophores can be used for labeling the probe sequence, including SIMA/HEX with BHQ1 quencher, ROX with BHQ2 quencher, TAMRA with BHQ2 quencher, and Texas Red/CalFluor 610 with BHQ2 quencher. Fluorescence signal can be detected using the Axxin T8 Isothermal Instrument, which can hold up to eight 200 ul tubes at a constant temperature. Two different fluorophores, with excitation ranging from 350 nm to 700 nm, can be read per tube, which can be any combination of FAM, HEX, or ROX, for multiplexing of reactions needed for assay optimization. The machine graphs the fluorescence signal over time, compares results from control and test channels, and gives negative or positive results based on customized algorithms that are built using initial average fluorescence, gradient, and threshold signal obtained from graphed data.

Primers and probes can be designed using Primer-BLAST and the most recent SARS-CoV-2 sequences for both Delta and Omicron variants. Several unique regions in the SARS-CoV-2 genome can be targeted, especially N, S, E, and RdRP genes. This is important for viral detection in the event that some mutations appear and cause some of the primers or probes to fail. Periodic BLAST searches aligning primer and probe sequences to published SARS-CoV-2 sequences ensures that they are still working.

Screening of optimal primer sets and probes (n=3 per gene) are done in a 200 μl test tube. Probes, as well as forward and reverse primers for N, S, E, RdRP, and RNAse P genes are added separately to a RT-RPA pellet (TwistAmp® Exo kit) that has been resuspended in rehydration buffer. RNAse P will be used as an extraction control for each reaction. The probe for N gene will be FAM-labeled and the probe for the RNAse P gene will be ROX-labeled. SARS-CoV-2 positive RNA controls diluted in human nasal solution are tested at three different concentrations (Zeptometrix, Inc.). Control tests are run with no template and no RT. The Axxin T8 Isothermal reader is used to read both FAM and ROX signals over a 30-60-minute period after addition of Magnesium Acetate at 37° C. This temperature is selected because it is optimal for both MMLV-RT and RPA.

Anticipated results from RPA reactions that have cDNA as a template or no template are exponential curves for FAM and ROX signals over time. The average time to threshold fluorescence signal can be measured for both genes within each reaction, and the experiment repeated three times. The goal is to select primer sets that give the fastest signal with compatible amplification curves for target genes and control RNAseP within each reaction at the lowest template RNA concentration. This is essential for use of these primers and probes in a multiplex fashion on AH-COV.

### 2. Determine AH-COV Assay Conditions

Once primer pairs and probes are selected, they can be used to prepare AH-COV-Unit-2-Fluor (FIG. 3C), wherein they will be dried on the chip along with the TwistAmp® Exo reaction mix. To measure assay LOD, five different concentrations of SARS-CoV-2 RNA control can be made and aliquoted into the sample port of five different AH-COV-Unit-2-Fluor. The fluorescence signal can be monitored using a fluorescence microscope. Time lapse images are able to assist in the analysis of the data and measurement of the average time to fluorescence. The lowest detectable concentration of virus using primers and probes for each of the target viral genes should be used to determine the LOD.

Using this embodiment, it is anticipated that one can observe the fluorescence signal as it appears down the channels of AH-COV-Unit-2-Fluor. The LOD is determined based on the time to signal appearance at the lowest RNA concentration. These results help determine the time needed to run the assay.

In other embodiments, AH-COV-Unit-2-Fluor is envisioned to have a variety of fluorescence probes that are designed to detect several genes and the signal can be detected after hybridization to a cDNA dot blot placed in the detection chamber. In this case, AH-COV-Fluor is mostly useful for assay optimization in a laboratory setting.

It is also possible to develop a smartphone-based reader for fluorescence signal detection and quantitative analysis of the results. This will require an LED light source for excitation combined with the appropriate emission filter.

### 3. Optimize AH-COV for At-Home Use

In another preferred embodiment, AH-COV-Unit-2-LF (FIG. 3B) is retrofitted with lateral flow strips for easy and rapid colorimetric detection of labeled signal and control using labeled primers in the Twist® Amp Basic amplification reaction mix on the device. Suitable RT-RPA primers are obtained from the above-mentioned primer design experiments and forward primers are labeled with biotin, while reverse primers are labeled with digoxigenin for SARS-CoV-2 genes and FITC/FAM for control genes, such as RNAseP. As a result, the amplified product is either labeled with Biotin+FITC/FAM (control), or Biotin+Digoxigenin, and is detected on the chip using lateral flow sandwich assays.

The lateral flow strip FIGS. 3B, 319 has a sample pad on one end and an absorbent pad FIGS. 3B, 318 on the other. Three different lines are made on this paper: one has anti-digoxigenin antibody, the second has anti-FAM antibody, and the third has biotin as a control. Upon introduction of a sample on AH-COV-Unit-2-LF, each amplified target is labeled with Biotin on the one end and digoxigenin on the other, while the control RNAse P is labeled with FITC/FAM and Biotin. These amplified products flow through the lateral flow membrane and bind to the antibodies.

This signal can be further amplified by using Streptavidin conjugated BioReady spherical gold nanoshells (Sigma) that bind to biotin. Unlike conventional gold nanoparticles which are 40 nm in diameter, the BioReady nanoshells are 150 nm with a streptavidin-bound gold shell surrounding a silica core. They are thought to enhance assay sensitivity by 20-fold and they appear as a gray line, as opposed to the pink line for gold on the lateral flow strip. In addition, they are cheaper than gold.

In samples that contain the target gene, three grey lines FIGS. 3B, 311 may appear on the nitrocellulose membrane. A line for RNAse P serves as a swab extraction control, showing that there are no issues with the NA extraction step. A line for biotin constitutes as a control for streptavidin-biotin binding on the nitrocellulose membrane, indicating success of the lateral flow experiment. Again, no sample and no RT controls are needed. As for the third line, it is for the target gene.

To determine sensitivity of AH-COV, LOD data is first obtained on Unit 2 by testing SARS-CoV-2 positive and negative control RNA (Zeptometrix, 10 μl) at five different dilutions in nasal fluid on AH-COV-Unit-2-LF. Next, LOD is determined on AH-COV when both units are put together starting from a sample swab dipped into these different RNA control solutions and transferred into either AH-COV-Unit-1-D or AH-COV-Unit-1-S. As described earlier, AH-COV-Unit-1-D docks directly into the sample port of AH-COV-Unit-2-LF and the sample mixes with the amplification reaction reagents, with the results appearing within 20-30 minutes on the lateral flow strip. For AH-COV-Unit-1-S, the dipstick is transferred from this unit and placed in the sample port of AH-COV-Unit-2-LF, triggering the downstream events for amplification, labeling, and target detection.

### 4. Determine Specificity of AH-COV

As per FDA guidelines, assay specificity is determined by testing for cross-reactivity with other viruses. This is done in two ways: either in silico by performing monthly BLAST analysis for the primer and probe sequences against all publicly available genomic sequences, or by using AH-COV to test a commercially available panel of thirteen inactivated bacteria/fungi and twenty viruses, including Flu-A, Flu-B, SARS-CoV-1, MERS-CoV, RSV-A, and RSV-B.

Upon successful completion of the analytical validity studies, clinical validity of the device needs to be determined when AH-COV-Unit-1 and AH-COV-Unit-2-LF are put together. Patient samples (30 SARS-CoV-2 positive and 30 negative) need to be tested for SARS-CoV-2 on the device and results compared to those obtained by qRT-PCR. SARS-CoV-2 negative samples, but positive for other viral infections as recommended by CDC also is tested to rule out cross-reactivity. The percent positive and negative results in COVID-19 patients and the percentage positive and negative results in COVID-19 negative patients is determined using AH-COV. ROC curves are graphed and the Area Under the Curve (AUC) is measured. This shows the assay power in correctly differentiating true positives from negative patients. Each test is performed by three different operators to determine reproducibility of the results obtained on AH-COV.

These studies demonstrate the clinical validity of the assembled AH-COV device by providing assay sensitivity and specificity. Upon completion of these experiments, Phase 2 studies can involve beta testing at two different clinics to determine ease of use of the device by patients, and correlate results with other COVID-19 assays. This can give all the data necessary for submission of the AH-COV device for FDA approval. The present invention is considered to be a low complexity device for home or POC use.

### EXAMPLES

The present invention is more particularly described in the following non-limiting examples, which are intended to be illustrative only, as numerous modifications and variations therein will be apparent to those skilled in the art.

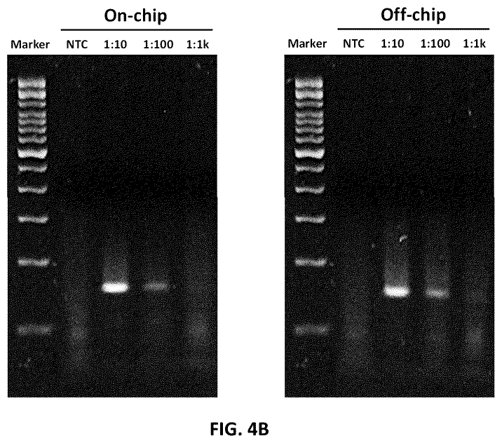

Several versions of Unit-2 were manufactured by soft lithography to determine optimal fluidics and amplification reaction conditions using TwistAmp® Basic chemistry. The first version of this unit, Unit-2.1, has one inlet and one outlet. Two different heights 30 μm (FIG. 4A, Left) or 100 μm (FIG. 4A, Right) were manufactured with SU-8. A pump was attached to the inlet tubing to push the fluid through the micro channels. Device loading and fluid flow was tested using red food coloring solution and the results showed better device loading in the 100 μm unit.

We then tested whether we can amplify control DNA on the 100 μm Unit-2.1 using the Twist®Amp Basic DNA amplification kit. For this experiment, we first performed the assay in a 0.5 ml Eppendorf tube (Off-chip; FIG. 4B, Right) according to manufacturer instructions, using three dilutions of the Positive Control DNA (1:10, 1:100, and 1:1000), and no template control (NTC). The reaction was incubated at 39° C. for 20 minutes, followed by ice. Since the Twist® Amp Basic kit does not label amplified DNA, we ran 5 μl from each reaction on a 2% agarose gel (Off-chip; FIG. 4B, Right), and observed the 143 bp amplification product in the 1:10, 1:100, but not 1:1000 dilutions of the Positive Control DNA.

We then performed the experiment using AH-COV-Unit-2.1; one unit for each DNA concentration and a NTC. One Twist® Amp Basic reaction tube containing the dried reaction mix, was rehydrated with 37.5 μl buffer, and divided into 4 aliquots. For each unit, MgOAc (0.63 μl) was added first, followed by reaction mix (9.37 μl) and the DNA (2.5 μl). Mixing of reagents was observed in the serpentine channels, while the devices were incubated in a sealed container for 20 minutes at 39° C. An aliquot of the product coming out of AH-COV-Unit-2 outlet was loaded on the agarose gel. Similar results were observed for On-chip (FIG. 4B, Left) and Off-chip (FIG. 4B, Right) experiments, showing feasibility of isothermal amplification of DNA on AH-COV.

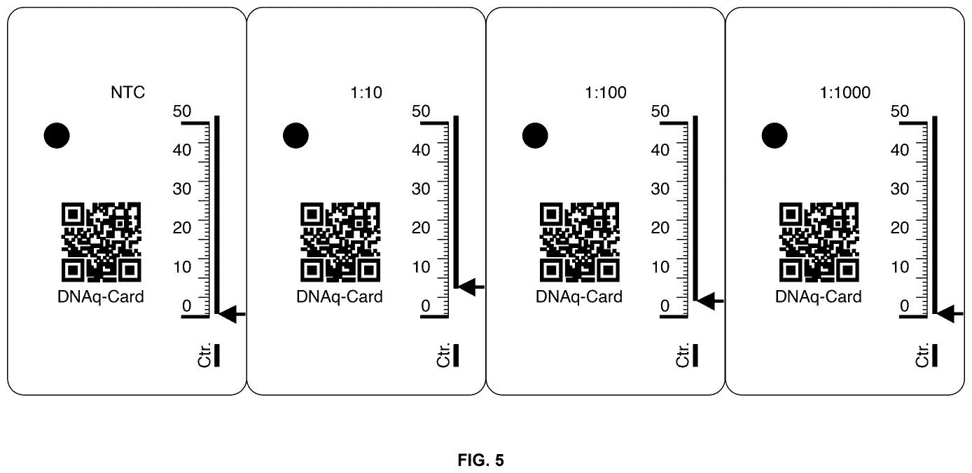

One of the challenges that we have experienced during the pandemic was the ability to obtain the Twist® Amp nfo kit, which is designed to work with lateral flow strips, a feature that gives a colorimetric assay readout. To overcome this supply chain shortage, we decided to use DNA-q Card™ from Accel Diagnostics (FIG. 5) to quantify the amount of RPA products obtained on the chip. DNA-q Card consists of a disposable microfluidic device for quantitation of dsDNA from PCR products without pre-purification. It has a 2-5 ng/ul LOD. For this experiment, four different AH-COV-Unit-2.1 were used to test control DNA at 1:10, 1:100, 1:1000 control DNA, and NTC. An aliquot of the reaction product (5 μl) was added to Buffer A (25 μl) from the q-Card kit, incubated for one minute at room temperature, and mixed with Buffer B (100 μl) by finger flicking for 30 seconds. The entire mix was then added to the DNAq-Card™ inlet, and a picture was taken of the card after 1 minute (FIG. 5)..

These cards work in such a way that dsDNA displaces a blue liquid on the card which is proportional to its concentration. A graduated line assists in making the final measurement for DNA concentration, which is done by the DNAq Mobile application using a smartphone. The preliminary results showed an undetectable signal in NTC, and 1.4, 6.1, and 10.7 ng/ul for starting control DNA at 1:1000, 1:100, and 1:10 dilutions, respectively. This suggested that the DNAq-Card™ can be an appropriate detection system which can be incorporated on AH-COV.

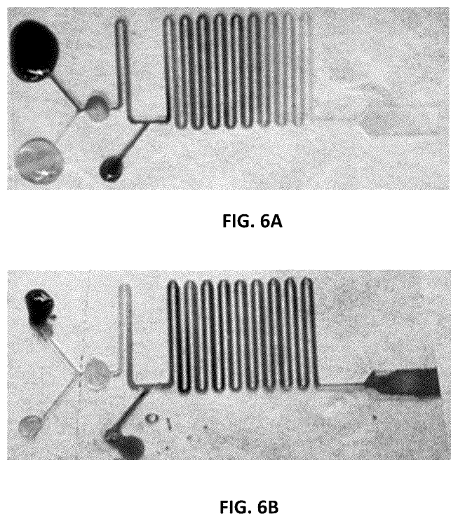

Another method of fabrication for Unit-2 was to make microfluidic patterns on the surface of hot laminating pouches (FIGS. 6A and 6B) using a Silhouette Curio cutter, peeling off the adhesive material for the channels, punching holes for the inlets and outlet, placing a piece of ShrinkFun in between, and running through a Fellows Venus 125 laminator. This device is called a AH-COV Unit 2.2. Inlets of this device can be retrofitted with self-adhesive vinyl furniture bumpers with a punched hole, where an AH-COV Unit 1 can dock.

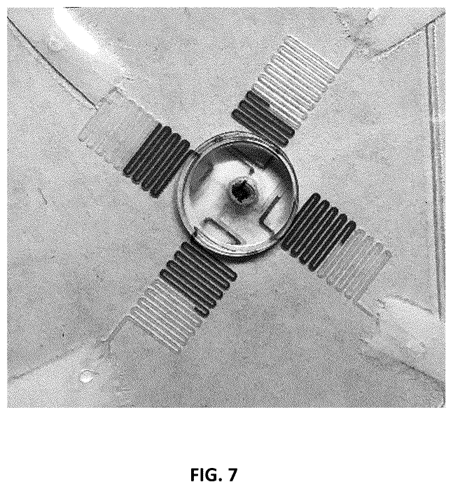

This manufacturing protocol allowed for the rapid prototyping of AH-COV Unit 2 and testing of a variety of scenarios for mixing of the reagents, including addition of sample RNA, RPA reaction mix, and Magnesium Acetate, separately. FIGS. 6A and 6B show how mixing of these reagents can occur without the need for a pump, which was required for AH-COV Unit 2.1. Furthermore, multiple reactions can be performed simultaneously on the same device, when several microfluidic channels are printed on the same device and linked to the same inlet (FIG. 7).

In summary, AH-COV can solve a major bottleneck in COVID testing during a pandemic, since it is a low complexity rapid test for SARS-CoV-2 detection. There are several innovative steps that make this device portable and easier to use than other currently available assays on the market. Basically, this device encapsulates all the reagents and steps needed for extraction and purification of RNA from sample swabs (AH-COV-Unit-1) to amplification and detection (AH-COV-Unit-2) without the need for complicated temperature controls or detection systems. This device can be used for either RNA or DNA detection and can be developed as a tool for rapid detection of all types of RNA or DNA biomarkers. There are at least three versions of this device including one that uses AH-COV-Unit-2-Fluor and is fluorescence-based requiring a fluorescent reader, and another that uses AH-COV-Unit-2-LF and is lateral flow-based making it optimal for use in any setting by someone who does not have any specialized training. Thus, AH-COV meets the need for rapid, accurate, and sensitive low complexity SARS-CoV-2 detection assays that can be self-administered at home, at POC, and in any setting.

While the invention has been particularly shown and described with reference to embodiments described above, it will be understood by those skilled in the art that various alterations in form and detail can be made therein without departing from the spirit and scope of the invention, as defined by the appended claims.

What is claimed is:

1. A microfluidic device referred to as Unit 2, comprising:
an inlet for sample delivery;
an inlet or reservoir for housing amplification reaction reagents and labeling reagents at constant temperature;
channels for mixing reagents;
channels for incubation and amplification of signal; and
a chamber and outlet for signal detection.

2. The microfluidic device of claim 1, wherein the microfluidic device comprises a transparent microfluidic slide prepared using well known soft lithographic methods.

3. The microfluidic device of claim 2, wherein a silhouette curio cutter is used to cut the adhesive, wherein a piece of ShrinkFun is placed between a top and bottom layer of a hot laminating pouch, wherein a Fellows Venus 125 laminator is used to fuse the pouch where the adhesive is present leaving behind patently open channels, and wherein holes are punched for inlets and outlets.

4. The microfluidic device of claim 3, wherein microfluidic patterns are created on a surface of the hot laminating pouch using a cutter that cuts an adhesive which is peeled off to reveal the channels prior to sealing off the pouch with a laminator.

5. The microfluidic device of claim 1, wherein the amplification reaction reagents are dried inside the reservoir or the channels inside Unit 2.

6. The microfluidic device of claim 5, wherein the amplification reaction reagents comprise enzymes and reagents needed for isothermal DNA synthesis comprising strand displacement amplification (sSDA), multiple displacement amplification (MDA), recombinase polymerase amplification (RPA), helicase dependent amplification (HDA), rolling circle amplification (RCA), or loop mediated isothermal amplification (LAMP).

7. The microfluidic device of claim 6, wherein RNA is amplified and labeled by either adding reverse transcriptase to convert RNA to complementary DNA (cDNA) followed by the isothermal DNA synthesis, or using replicase (RdRP) which can catalyze de novo RNA synthesis in a primer dependent fashion.

8. The microfluidic device of claim 1, wherein the labeling reagents are a combination of: SIMA/HEX/BHQ1, ROX/BHQ2, TAMRA/BHQ2, or TEXAS RED/CalFluor 610/BHQ2 probes, or digoxigenin, biotin, FITC/FAM labeled primers, d-ATP, nucleosides, molecular beacons, intercalating dyes, and combinations thereof.

9. The microfluidic device of claim 1, wherein an amplified product is collected at the end of the reaction for further downstream testing by gel electrophoresis, DNAq-Cards™ (Accell Dx), or lateral flow strips comprising PCRD (Abingdon Health), Hybridetect (Milennia), or U-Star (Twist Dx).

10. The microfluidic device of claim 1, wherein fluorescently labeled targets are imaged in real-time using fluorescence microscopy, or fluorometry comprising Axxin T8 Isothermal reader.

11. The microfluidic device of claim 1, wherein the detection chamber is retrofitted with a cDNA dot blot, and wherein the detection chamber is imaged with a smartphone-based reader for fluorescence signal detection and measurement which requires an LED light source for excitation combined with the appropriate emission filter.

12. The microfluidic device of claim 9, wherein the amplified product is either labeled with Biotin+FITC/FAM or Biotin+Digoxigenin, and the signal is detected using a lateral flow sandwich assay.

13. The device of claim 12, wherein a lateral flow strip of the lateral flow sandwich assay has a sample pad on one end and an absorbent pad on the other with three lines made on the paper, wherein the first line is for anti-digoxigenin antibody, the second line is for anti-FAM antibody, and the third line is for biotin as a control, wherein each amplified product can be labeled with Biotin on the one end and digoxigenin on the other, wherein the control has FITC/FAM and Biotin labels, wherein the amplified product exiting from the serpentine channels flows through the membrane and binds to the antibodies, wherein presence of colored deposit on each line indicates binding and detection of a target gene.

14. The device of claim 13, wherein the signal is further amplified by using Streptavidin conjugated conventional gold nanoparticles that bind to biotin and produces a pink line.

15. The device of claim 14, wherein the signal further is amplified with streptavidin conjugated to 150 nm BioReady spherical gold nanoshells surrounding a silica core, or any iteration of streptavidin conjugated gold particles that enhance assay sensitivity and appear as a gray line on the lateral flow strip.
